(19) United States
(12) Patent Application Publication
(10) Pub. No.: US 2023/0167064 A1
(43) Pub. Date: Jun. 1, 2023

(54) TREATMENTS OF INFLAMMATORY BOWEL DISEASE

(71) Applicant: Aqilion AB, Helsingborg (SE)

(72) Inventor: Martin Hans Johansson, Helsingborg (SE)

(73) Assignee: Aqilion AB, Helsingborg (SE)

(21) Appl. No.: 17/922,043

(22) PCT Filed: Apr. 30, 2021
(86) PCT No.: PCT/EP2021/061462

§ 371 Date: Oct. 28, 2022

(30) Foreign Application Priority Data
Apr. 30, 2020 (GB) 2006390.5

(51) Int. Cl. C07D 215/227; A61K 45/06; A61P 1/04; A61P 29/00

(52) U.S. Cl. CPC C07D 215/227; A61K 45/06; A61P 1/04; A61P 29/00

(57) ABSTRACT

The invention provides a compound according to formula (I); Formula (I) as more particularly defined in the specification, or a pharmaceutically acceptable salt or solvate thereof for use in the treatment or prophylaxis of an inflammatory bowel disease. The invention also provides pharmaceutical compositions comprising a compound of the invention and methods of treatment using a compound of the invention.

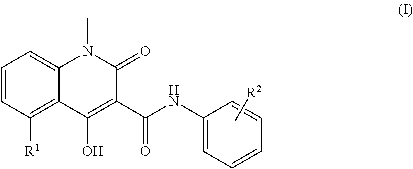

### FIELD OF THE INVENTION

The present invention relates to the treatment and/or prophylaxis of inflammatory bowel diseases. The present invention also relates to dosage regimens and kits that find utility in the treatment and/or prophylaxis of inflammatory bowel diseases.

### BACKGROUND OF THE INVENTION

Inflammatory Bowel Diseases (IBDs) are a heterogeneous group of diseases and disorders that are characterised by chronic inflammation of the gastrointestinal wall. Symptoms caused by the chronic inflammation include abdominal pain, diarrhoea, general feeling of ill health, and loss of appetite and poor absorption of nutrients from food, which often leads to weight loss. There are two main forms of IBD: ulcerative colitis (UC), which typically begins in the descending colon and rectum and may extend continuously to involve the entire colon (pancolitis), and Crohn's disease (CD), which most commonly involves the ileum and ascending colon. Indeterminate Colitis (IC) may also be considered a form of IBD. An IBD is categorised as IC when the disease state is indistinguishable from CD and UC.

Currently-available treatments for IBDs are largely directed to the reduction of symptoms and the maintenance of remission. To prolong remission, long-term maintenance therapy is often required. First-line treatment often involves the use of aminosalicylates and/or corticosteroids. Second-line treatments include immunosuppressants, Tumor Necrosis Factor (TNF) inhibitors and integrin inhibitors. Second-line treatments may be used as a monotherapy, or in combination with one or more first or second-line treatments. Often, surgical intervention is required.

Immunomodulatory drugs have also shown promise for the treatment of IBDs. N-alkyl 1,2-dihydro-4-hydroxy-2-oxo-quinoline-3-carboxanilides (hereinafter referred to as N-alkyl quinoline-3-carboxanilides) are one particularly promising class of compounds that have been shown to have immunomodulatory properties. The immunomodulatory properties and therapeutic potential of N-alkyl quinoline-3-carboxanilides was first reported in the 1980s (for example, see U.S. Pat. No. 4,547,511). One member of this class is laquinimod, which has been reported to be beneficial for the treatment of Crohn's Disease (see for example, D'Haens et al., Gut. 2015, 64(8):1227-35, and WO 2011/014255).

Despite initial promise, poor efficacy, toxicity and instability have limited the clinical success of N-alkyl quinoline-3-carboxanilides. For example, Jansson et al., (J. Org. Chem. 2006, 71, 1658-1667) reported that N-alkyl quinoline-3-carboxanilides are chemically reactive towards nucleophiles, making them unstable in their neutral form. N-alkyl quinoline-3-carboxanilides, such as laquinimod, have also been shown to be readily metabolised by cytochrome P450 (CYPs) enzymes into various active metabolites, potentially having differing potencies, toxicities, and physicochemical properties (for example, see Tuvesson et al., 2005, Drug Metab. Dispos, 33:866-872, 2005). Notably, laquinimod was refused market authorisation in Europe for the treatment of relapsing remitting multiple sclerosis due to concerns over human safety and poor efficacy (EMA 2014 Public Assessment Report—EMA/451905/2014).

Unfortunately, for many patients, available treatments for IBDs are ineffective at reducing symptoms and slowing disease progression. Many of the available treatments also cause serious adverse effects, such as an increased risk of infections, inflammation of the liver, nausea and sickness, weight gain, and in rare cases progressive multifocal leukoencephalopathy. There is therefore a significant clinical need for more effective therapeutic and prophylactic treatments for IBDs. In particular, there is a need for effective treatments that provide clinical benefits for patient suffering from an IBD, whilst also displaying manageable side-effects.

### SUMMARY OF THE INVENTION

The present invention provides a compound of formula (I),

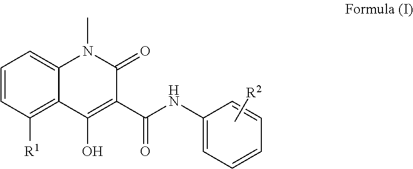

or a tautomer thereof,

wherein,

R¹ is hydrogen, hydroxyl, C₁₋₃alkyl, OC₁₋₃alkyl or halogen; and

R² is hydrogen, hydroxyl, OC₁₋₃alkyl, halogen or C₁₋₃alkyl optionally substituted with at least one halogen;

or a pharmaceutically acceptable salt or solvate thereof;

for use in the treatment and/or prophylaxis of an inflammatory bowel disease.

The present invention also provides a pharmaceutical composition comprising a compound of formula (I) and at least one pharmaceutically acceptable excipient, wherein said pharmaceutical composition has a solid or semi-solid form adapted for release of the compound in the small and/or large intestine. The composition may optionally comprise one or more additional therapeutic agents.

The present inventors have found that the compounds for use and the compositions according to the present invention are surprisingly effective for the treatment and/or prophylaxis of inflammatory bowel diseases, such as Crohn's disease and Ulcerative Colitis. The present inventors have found that compounds of formula (I) are surprisingly effective at reducing inflammation/edema associated with inflammatory bowel disease in an in vivo inflammatory bowel disease mouse model and at preventing the development of Ulcerative Colitis in an in vivo Ulcerative Colitis mouse model. The efficacy of the compounds of formula (I) allows for their use in effective therapeutic treatments for reducing the symptoms of inflammatory bowel diseases, in particular Crohn's disease and Ulcerative Colitis, and prolonging disease remission.

The present invention further provides a method of treating and/or preventing an inflammatory bowel disease, said method comprising administering a pharmaceutically effective amount of a compound of formula (I), or pharmaceutical composition of the present invention to a subject suffering from, or at risk of developing, an inflammatory bowel disease.

The present invention also provides a method of delivering a compound of formula (I), or pharmaceutical composition of the invention, to the small and/or large intestine of a subject suffering from, or at risk of developing, an inflammatory bowel disease.

Also provided herein is a use of a compound of formula (I) in the manufacture of a medicament for the treatment and/or prophylaxis of an inflammatory bowel disease. The present invention further provides a kit comprising a compound according to the present invention, together with one or more pharmaceutically acceptable excipient, and optionally one or more further therapeutic agent. The kits of the present invention find use in the treatment and/or prophylaxis of inflammatory bowel diseases.

### DESCRIPTION OF FIGURES

FIG. 1 shows the colitis score measured in mice in a DSS model of Ulcerative Colitis using C57Bl/6 mice. Mice that received example compound 1 displayed a lower colitis score compared to mice that received vehicle only (CMC-Na, 2% w/v), or an anti-TNFα antibody.

FIG. 2 shows the body weight change (% change from day 1 of experiment) of C57Bl/6 in the DSS model of Ulcerative Colitis. Mice that received example compound 1 displayed reduced weight loss compared to mice that received vehicle only (CMC-Na, 2% w/v), or an anti-TNFα antibody.

FIG. 3 shows the colon length (cm) in mice in the DSS mouse model of Ulcerative Colitis at Day 10 of the experiment. Mice that received example compound 1 were found to have greater colon length compared to mice that received vehicle only (CMC-Na, 2% w/v), or an anti-TNFα antibody.

FIG. 4 shows the change in body weight in groups of mice after treatment with Example compound 2 in a mouse model of inflammatory bowel disease.

FIGS. 5 and 6 show markers of inflammation in mice treated with Example compound 2 in a mouse model of inflammatory bowel disease.

FIG. 7 shows the level of Example compound 2 in the plasma at the various time points in an in vivo pharmacokinetic study.

### DETAILED DESCRIPTION OF THE INVENTION

The present inventors have found that particular N-desalkyl quinoline-3-carboxanilides show surprising efficacy in treating or preventing the symptoms and development of inflammatory bowel diseases. In particular, the present inventors have found that N-desalkyl quinoline-3-carboxanilides according to formula (I) display surprisingly beneficial properties for the treatment or prophylaxis of IBDs, such as Crohn's disease and Ulcerative Colitis.

As discussed in more detail below, the present inventors have found that 5-chloro-4-hydroxy-1-methyl-2-oxo-N-phenyl-quinoline-3-carboxamide (DELAQ) reduced weight loss and had a protective effect against the development of the clinical symptoms of Ulcerative Colitis in an in vivo mouse model of Ulcerative Colitis. The inventors also found that DELAQ was effective at reducing colon shortening which indicates a reduction in edema associated with Ulcerative Colitis. In a further study using the potassium salt of DELAQ, the present inventors found that DELAQ was effective in inhibiting inflammation/edema associated with inflammatory bowel disease, as evaluated in a CD4⁺ adoptive transfer induced inflammatory bowel disease in mice. Furthermore, mice treated orally with the potassium salt of DELAQ had a significant increase in CYP1A1 mRNA expression compared to untreated animals indicating that the compound is effective in activating the aryl hydrocarbon receptor (AhR) in the colon. The surprising efficacy of DELAQ at reducing inflammation allows for its use in effective treatment and prophylaxis of IBDs, such as Crohn's disease and Ulcerative Colitis. In some embodiments, the efficacy and other properties of the compound allow for it to be efficacious when dosed orally.

N-Desalkyl Quinoline-3-Carboxanilides

N-desalkyl quinoline-3-carboxanilides have previously been reported to be active metabolites of N-alkyl quinoline-3-carboxanilides such as laquinimod and tasquinimod. In isolated form, N-desalkyl quinoline-3-carboxanilides have been reported to be unsuitable for in vivo administration due to poor stability and low aqueous solubility (for example, see Tuvesson et al., 2005, Drug Metab. Dispos, 33:866-872, 2005, WO 2012/050500 and Mariout et al., 2017, Tox. Appl. Pharm., 326, 54-65).

However, the present inventors have found that N-desalkyl quinoline-3-carboxanilides of formula (I) are surprisingly effective at treating and preventing IBDs, in particular CD and UC. As such, the present invention provides compounds according to formula (I):

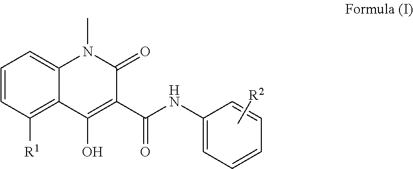

for use in the treatment or prophylaxis of an inflammatory bowel disease, in particular CD and UC.

In the compound of formula (I), R¹ may be hydrogen, hydroxyl, C₁₋₃alkyl, OC₁₋₃alkyl or halogen. Preferably, R¹ is selected from hydrogen, hydroxyl, methyl, ethyl, OCH₃, OCH₂CH₃, F, Cl, Br and I. More preferably, R¹ is selected from hydrogen, ethyl, OCH₃, F and Cl. Even more preferably, R¹ is selected from hydrogen, ethyl, OCH₃ and Cl.

In the compounds of formula (I), R² may be hydrogen, hydroxyl, OC₁₋₃alkyl, halogen or C₁₋₃alkyl optionally substituted with at least one halogen (for example monohaloC₁₋₃alkyl, dihaloC₁₋₃alkyl and trihaloC₁₋₃alkyl), wherein halogen is selected from F, Cl, Br and I. Preferably, R² is selected from hydrogen, hydroxyl, C₁₋₃alkyl, OCH₃, OCH₂CH₃, F, Cl, or C₁₋₃alkyl substituted with at least one F or Cl (for example monohaloC₁₋₃alkyl, dihaloC₁₋₃alkyl and trihaloC₁₋₃alkyl). More preferably, R² is selected from hydrogen, hydroxyl, methyl, ethyl, monofluoromethyl, difluoromethyl, trifluoromethyl, monochloromethyl, dichloromethyl and trichloromethyl. Even more preferably, R² is selected from hydrogen, monofluoromethyl, difluoromethyl and trifluoromethyl. Even more preferably, R² is hydrogen or trifluoromethyl.

In a preferred embodiment, in the compounds of formula (I), R¹ is selected from hydrogen, OCH₃ and ethyl, and R² is selected from hydrogen and trifluoromethyl. For example, R¹ may be hydrogen and R² may be hydrogen or trifluoromethyl. For example, R¹ may be OCH₃ and R² may be hydrogen or trifluoromethyl. For example, R¹ may be ethyl and R² may be hydrogen or trifluoromethyl.

In certain embodiments, in the compounds of formula (I), R² is not trichloromethyl when R¹ is OCH₃.

In certain preferred embodiments, in the compounds of formula (I), R² is hydrogen.

In preferred embodiments, the compound for use according the present invention is a compound of formula (Ia):

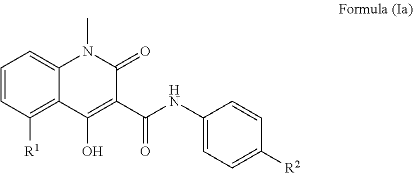

In one preferred embodiment, the compound of formula (I) is selected from the group consisting of:

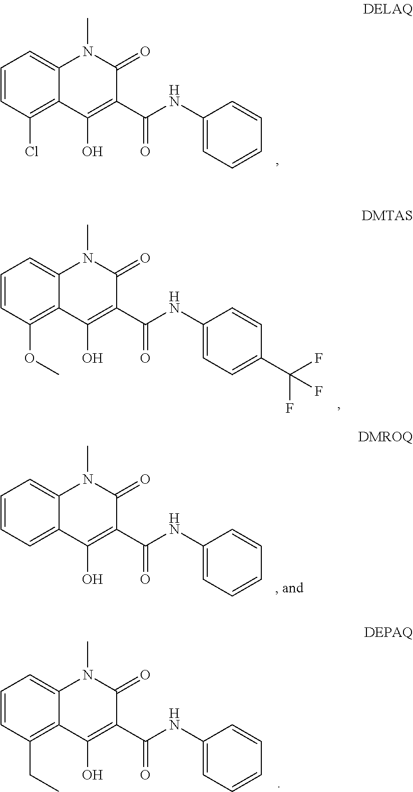 [SMILES: Cn1c(=O)c(C(=O)Nc2ccccc2)c(O)c2c(Cl)cccc21 ; COc1cccc2c1c(O)c(C(=O)Nc1ccc(C(F)(F)F)cc1)c(=O)n2C ; Cn1c(=O)c(C(=O)Nc2ccccc2)c(O)c2ccccc21 ; CCc1cccc2c1c(O)c(C(=O)Nc1ccccc1)c(=O)n2C]

In another preferred embodiment, the compound of formula (I) is DELAQ.

For the avoidance of doubt, in this document, it is intended that compounds of formula (I) include all tautomeric forms, salts and solvates thereof, unless stated otherwise.

The compounds for use according to the invention may be prepared using methods known to those skilled in the art of organic chemistry. Specific methods for preparing certain compounds according to the invention are described herein in the Examples section.

Depending upon the substituents present in compounds of formula (I), the compounds may form esters, amides, carbamates and/or salts. Salts of compounds of formula (I), which are suitable for use in the present invention, are those wherein a counterion is pharmaceutically acceptable. However, salts having non-pharmaceutically acceptable counter-ions are within the scope of the present invention, for example, for use as intermediates in the preparation of the compounds of formula (I) and their pharmaceutically acceptable salts, and physiologically functional derivatives. Suitable salts for use according to the invention include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Suitable cations which may be present in salts include alkali metal cations, such as sodium, potassium and calcium, and ammonium or amino cations.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} ed R. Lui CRC Press, page 553 and Byrn et al Pharm Res 12(7), 1995, 945-954. Before it is made up in solution, the compound of formula (I) may be in the form of a solvate. Solvates of compounds of formula (I) which are suitable for use as a medicament according to the invention are those wherein the associated solvent is pharmaceutically acceptable. For example, a hydrate is a pharmaceutically acceptable solvate.

The compounds of formula (I) may be crystalline or non-crystalline. Certain compounds of the invention may have more than one polymorphic form.

Pharmaceutical Compositions

While it is possible for a compound of formula (I) to be administered alone, it is preferable for it to be present in a composition and particularly in a pharmaceutical composition. Pharmaceutical compositions of the present invention comprise a compound of formula (I) and one or more pharmaceutically acceptable excipient. Pharmaceutical compositions include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration, although the most suitable route may depend upon, for example, the type of IBD that is under treatment.

Pharmaceutical compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The compound of formula (I) may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Pharmaceutical compositions for rectal administration may be presented as a suppository with carriers such as cocoa butter, synthetic glyceride esters or polyethylene glycol. Such carriers are typically solid at ordinary temperatures, but liquefy and/or dissolve in the rectal cavity to release the drug.

Certain compounds are known that, under suitable conditions, for example in the human body, can be converted into compounds of formula (I) by de-alkylation or by hydrolysis. Such compounds are referred to herein as precursors of a compound of formula (I).

In preferred embodiments, the composition of the present invention contains less than 10% mole percent (mol %) of a precursor of a compound of formula (I), wherein mol % is understood to be the proportion of compound present in the composition relative to the total combined number of moles of a compound of formula (I) and a precursor of a compound of formula (I) present in the composition. Preferably, a precursor is present in a composition of the invention at an amount of less than 5 mol %. For example, less than 4, 3, 2 or 1 mol % (for example, less than 0.9, 0.8, 0.7, 0.6, 0.5, 0.4, 0.3, 0.2 or 0.1 mol %) of a precursor.

Preferably, a precursor of a compound of formula (I) is present in the composition of the invention at an amount of less than 10 wt %, wherein wt % is understood to be the proportion of compound present in the composition relative to the total combined mass of a compound of formula (I) and a precursor of a compound of formula (I). More preferably, a precursor of a compound of formula (I) is present in the composition of the invention at an amount of less than 5 wt %. For example, less than 4, 3, 2 or 1 wt % (for example, less than 0.9, 0.8, 0.7, 0.6, 0.5, 0.4, 0.3, 0.2 or 0.1 wt %) of a precursor.

More preferably, the composition of the invention is substantially free from a precursor of a compound of formula (I).

In certain embodiments, the precursor of a compound of formula (I) is a compound of formula (II):

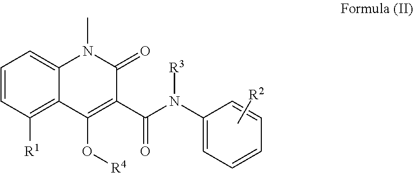

For the avoidance of doubt, in this document, it is intended that compounds of formula (II) include all tautomeric forms, salts and solvates thereof, unless stated otherwise.

In the compound of formula (II), R¹ may be hydrogen, hydroxyl, C₁₋₃alkyl, OC₁₋₃alkyl or halogen. Preferably, R¹ is selected from hydrogen, hydroxyl, methyl, ethyl, OCH₃, OCH₂CH₃, F, Cl, Br and I. More preferably, R¹ is selected from hydrogen, ethyl, OCH₃, F and Cl. Even more preferably, R¹ is selected from hydrogen, ethyl, OCH₃ and Cl.

In the compounds of formula (II), R² may be hydrogen, hydroxyl, OC₁₋₃alkyl, halogen or a C₁₋₃alkyl optionally substituted with at least one halogen (for example, monohaloC₁₋₃alkyl, dihaloC₁₋₃alkyl and trihaloC₁₋₃alkyl), wherein halogen is selected from F, Cl, Br and I. Preferably, R² is selected from hydrogen, hydroxyl, C₁₋₃alkyl, OCH₃, OCH₂CH₃, F, Cl, or C₁₋₃alkyl substituted with at least one F or Cl (for example monohaloC₁₋₃alkyl, dihaloC₁₋₃alkyl and trihaloC₁₋₃alkyl). More preferably, R² is selected from hydrogen, hydroxyl, methyl, ethyl, monofluoromethyl, difluoromethyl, trifluoromethyl, monochloromethyl, dichloromethyl and trichloromethyl. Even more preferably, R² is selected from hydrogen, monofluoromethyl, difluoromethyl and trifluoromethyl. Even more preferably, R² is hydrogen or trifluoromethyl.

In the compounds of formula (II), R³ and R⁴ may independently be selected from hydrogen, C(O)H, C(O)methyl, C(O)ethyl, C(O)propyl, C(O)CH(CH₃)₂, C(O)C(CH₃)₃, C(O)phenyl, C(O)CH₂phenyl, CO₂H, CO₂CH₃, CO₂CH₂CH₃, CO₂CH₂phenyl, C(O)NHCH₃, C(O)N(CH₃)₂, C(O)NHCH₂CH₃, C(O)N(CH₂CH₃)₂, C(O)NHphenyl, C(O)NHCH₂phenyl, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising methyl, ethyl, OCH₃, OCH₂CH₃, SCH₃, S(O)CH₃, S(O)₂CH₃, S(O)₂N(CH₃)₂, CF₃, OCF₃, F, Cl, OH, CO₂H, CO₂CH₃, CO₂CH₂CH₃, C(O)NH₂, C(O)N(CH₃)₂, NH₂, NH³⁺, N(CH₃)₂, NCH₃³⁺, NHC(O)CH₃, NC(═NH)NH₂, OS(O)₂OH, S(O)₂OH, OP(O)(OH)₂, and P(O)(OH)₂, provided that R³ and R⁴ are not both hydrogen; or wherein R³ is hydrogen, R⁴ may be P(O)(OH)₂, P(O)(OCH₃)₂, P(O)(OCH₂CH₃)₂, P(O)(OPhenyl)₂, P(O)(OCH₂Phenyl)₂, S(O)₂OH, S(O)₂NH₂ or S(O)₂N(CH₃)₂.

In the compounds of formula (II), R³ and R⁴ may independently be selected from hydrogen, C₁₋₂₀alkyl (for example, methyl and ethyl) and C₁₋₂₀alkylcarbonyl, said alkyl being linear or branched and optionally comprising one or more heteroatom (for example, N, O, S and P) and/or optionally substituted with one or more heteroatom (for example, N, O, S and P), C₆₋₁₀aryl and halogen (for example, F and Cl), provided that R³ and R⁴ are not both hydrogen.

In one embodiment, in the compound of formula (II), R³ is hydrogen and R⁴ is C(O)CH₃, R³ is C(O)CH₃ and R⁴ is hydrogen, or R³ and R⁴ are each C(O)CH₃.

In another embodiment, in the compound of formula (II), R¹ is hydrogen, ethyl, OCH₃ or Cl, R² is hydrogen or trifluroC₁₋₃alkyl, R³ is hydrogen, and R⁴ is C(O)CH₃. For example, R¹ is hydrogen, ethyl, OCH₃ or Cl, R² is hydrogen or trifluoromethyl, R³ is hydrogen, and R⁴ is C(O)CH₃.

In another embodiment, in the compound of formula (II), R¹ is hydrogen, ethyl, OCH₃ or Cl, R² is hydrogen or trifluroC₁₋₃alkyl, R³ is C(O)CH₃ and R⁴ is H. For example, R¹ is hydrogen, ethyl, OCH₃ or Cl, R² is hydrogen or trifluoromethyl, R³ is C(O)CH₃ and R⁴ is hydrogen.

In another embodiment, in the compound of formula (II), R¹ is hydrogen, ethyl, OCH₃ or Cl, R² is hydrogen or trifluroC₁₋₃alkyl, R³ and R⁴ are C(O)CH₃. For example, R¹ is hydrogen, ethyl, OCH₃ or Cl, R² is hydrogen or trifluoromethyl, R³ and R⁴ are C(O)CH₃.

In certain preferred embodiments, in a compound of formula (II), R³ is C₁₋₂₀alkyl optionally comprising one or more heteroatoms, and R⁴ is hydrogen. For example, R³ is C₁₋₂₀alkyl, C₁₋₁₆alkyl, C₁₋₁₄alkyl, C₁₋₁₀alkyl or C₁₋₆alkyl optionally comprising one or more heteroatoms. Preferably, R³ is methyl, ethyl or propyl and R⁴ is hydrogen.

More preferably, R³ is methyl or ethyl and R⁴ is hydrogen.

In one preferred embodiment, in the compound of formula (II), R¹ is hydrogen, ethyl, OCH₃ or Cl, R² is hydrogen or trifluroC₁₋₃alkyl, R³ is methyl, ethyl or propyl, and R⁴ is hydrogen. Preferably, R¹ is hydrogen, ethyl, OCH₃ or Cl, R² is hydrogen or trifluoromethyl, R³ is methyl or ethyl, and R⁴ is hydrogen. For example, R¹ may be Cl, R² may be trifluoromethyl, R³ may be methyl, and R⁴ may be hydrogen. For example, R¹ and R² may both be hydrogen, R³ may be methyl, and R⁴ may be hydrogen. For example, R¹ may be ethyl, R² may hydrogen, R³ may be ethyl, and R⁴ may be hydrogen.

In particularly preferred embodiments, R¹ and R² of a compound of formula (II) present in a composition of the invention are the same as R¹ and R² of a compound of formula (I) present in said composition of the invention.

In certain embodiments, the compound of formula (II) is the compound of formula (IIa):

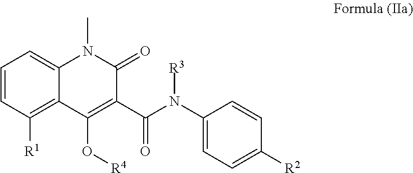

For the avoidance of doubt, in this document, it is intended that compounds of formula (IIa) include all tautomeric forms, salts and solvates thereof, unless stated otherwise.

In embodiments wherein a composition of the invention comprises DELAQ, the compound of formula (II) is laquinimod:

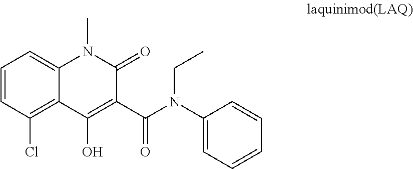 [SMILES: CCN(C(=O)c1c(O)c2c(Cl)cccc2n(C)c1=O)c1ccccc1]

Preferably, compositions of the invention comprising DELAQ, contain laquinimod at an amount of less than 10 mole percent (mol %) of the total combined number of moles of DELAQ and laquinimod present in the composition. More preferably, compositions comprising DELAQ contain laquinimod at amount of less than 5 mol %. For example, less than 4, 3, 2 or 1 mol % (for example, less than 0.9, 0.8, 0.7, 0.6, 0.5, 0.4, 0.3, 0.2 or 0.1 mol %) of laquinimod. Even more preferably, the composition of the invention is substantially free from laquinimod.

Preferably, compositions of the invention comprising DELAQ, contain laquinimod at an amount of less than 10 wt % of the total combined mass of DELAQ and laquinimod present in the composition. More preferably, laquinimod is present in the composition of the invention at an amount of less than 5 wt %. For example, less than 4, 3, 2 or 1 wt % (for example, less than 0.9, 0.8, 0.7, 0.6, 0.5, 0.4, 0.3, 0.2 or 0.1 wt %) of laquinimod. Even more preferably, the composition of the invention is substantially free from laquinimod.

In embodiments wherein the composition comprises DMTAS, the compound of formula (II) is tasquinimod:

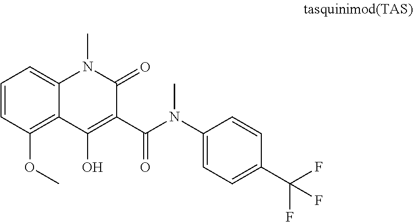 [SMILES: COc1cccc2c1c(O)c(C(=O)N(C)c1ccc(C(F)(F)F)cc1)c(=O)n2C]

Preferably, compositions of the invention comprising DMTAS, contain tasquinimod at an amount of less than 10 mole percent (mol %) of the total combined number of moles of DMTAS and tasquinimod present in the composition. More preferably, compositions comprising DMTAS contain tasquinimod at amount of less than 5 mol %. For example, less than 4, 3, 2 or 1 mol % (for example, less than 0.9, 0.8, 0.7, 0.6, 0.5, 0.4, 0.3, 0.2 or 0.1 mol %) of tasquinimod. Even more preferably, the composition of the invention is substantially free from tasquinimod.

Preferably, compositions of the invention comprising DMTAS, contain tasquinimod at an amount of less than 10 wt % of the total combined mass of DMTAS and tasquinimod present in the composition. More preferably, tasquinimod is present in the composition of the invention at an amount of less than 5 wt %. For example, less than 4, 3, 2 or 1 wt % (for example, less than 0.9, 0.8, 0.7, 0.6, 0.5, 0.4, 0.3, 0.2 or 0.1 wt %) of tasquinimod. Even more preferably, the composition of the invention is substantially free from tasquinimod.

In embodiments wherein the composition comprises DMROQ, the compound of formula (II) is roquinimex:

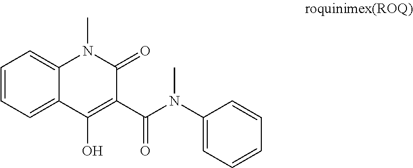 [SMILES: CN(C(=O)c1c(O)c2ccccc2n(C)c1=O)c1ccccc1]

Preferably, compositions of the invention comprising DMROQ, contain roquinimex at an amount of less than 10 mole percent (mol %) of the total combined number of moles of DMROQ and roquinimex present in the composition. More preferably, compositions comprising DMROQ contain roquinimex at amount of less than 5 mol %. For example, less than 4, 3, 2 or 1 mol % (for example, less than 0.9, 0.8, 0.7, 0.6, 0.5, 0.4, 0.3, 0.2 or 0.1 mol %) of roquinimex. Even more preferably, the composition of the invention is substantially free from roquinimex.

Preferably, compositions of the invention comprising DMROQ, contain roquinimex at an amount of less than 10 wt % of the total combined mass of DMROQ and roquinimex present in the composition. More preferably, roquinimex is present in the composition of the invention at an amount of less than 5 wt %. For example, less than 4, 3, 2 or 1 wt % (for example, less than 0.9, 0.8, 0.7, 0.6, 0.5, 0.4, 0.3, 0.2 or 0.1 wt %) of roquinimex. Even more preferably, the composition of the invention is substantially free from roquinimex.

In embodiments wherein the composition comprises DEPAQ, the compound of formula (II) is paquinimod:

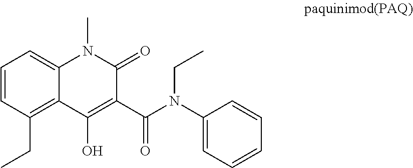 [SMILES: CCc1cccc2c1c(O)c(C(=O)N(CC)c1ccccc1)c(=O)n2C]

Preferably, compositions of the invention comprising DEPAQ, contain paquinimod at an amount of less than 10 mole percent (mol %) of the total combined number of moles of DEPAQ and paquinimod present in the composition. More preferably, compositions comprising DEPAQ contain paquinimod at amount of less than 5 mol %. For example, less than 4, 3, 2 or 1 mol % (for example, less than 0.9, 0.8, 0.7, 0.6, 0.5, 0.4, 0.3, 0.2 or 0.1 mol %) of paquinimod. Even more preferably, the composition of the invention is substantially free from paquinimod.

Preferably, compositions of the invention comprising DEPAQ, contain paquinimod at an amount of less than 10 wt % of the total combined mass of DEPAQ and paquinimod present in the composition. More preferably, paquinimod is present in the composition of the invention at an amount of less than 5 wt %. For example, less than 4, 3, 2 or 1 wt % (for example, less than 0.9, 0.8, 0.7, 0.6, 0.5, 0.4, 0.3, 0.2 or 0.1 wt %) of roquinimex. Even more preferably, the composition of the invention is substantially free from paquinimod.

For the avoidance of doubt, in this document, when the mol % of a compound of formula (I) or (II) is provided, it is understood to be the total combined number of moles of the compound of formula (I) and (II) present in the composition. For example, in a composition containing a total of 100 mmol of the compound of formula (I) and (II), and wherein the compound of formula (II) is present at 10 mol %, said composition contains 90 mmol of a compound of formula (I) and 10 mmol of a compound of formula (II). As a further example, in a composition of the invention containing a total of 100 μmol of DELAQ and laquinimod, and wherein less than 10 mol % is laquinimod, said composition contains less than 10 μmol of laquinimod (i.e. less than 3.6 mg of laquinimod, excluding the mass of any counterion or solvent present) and more than 90 μmol of DELAQ (i.e. more than 29.5 mg of DELAQ, excluding the mass of any counterion or solvent present).

In addition, in this document, when the wt % of a compound of formula (I) or (II) is provided, it is understood to be the to be the proportion relative to the total combined mass of a compound of formula (I) and compound of (II) present in the composition. For example, in a composition containing a total of 1000 mg of the compound of formula (I) and (II), and wherein the compound of formula (II) is present at 10 wt %, said composition contains 900 mg of a compound of formula (I) and 100 mg of a compound of formula (II). As a further example, in a composition of the invention containing a total of 1000 mg of DELAQ and laquinimod (excluding the mass of any counterion or solvent present), and wherein less than 10 wt % is laquinimod, said composition contains less than 100 mg of laquinimod (excluding the mass of any counterion or solvent present) and more than 900 mg of DELAQ (excluding the mass of any counterion or solvent present).

In preferred embodiments, a composition of the invention consists essentially of a compound of formula (I) and at least one pharmaceutically acceptable excipient.

It should be understood that in addition to the ingredients particularly mentioned above, the compositions for use in this invention may include other agents conventional in the art having regard to the type of composition in question.

The compositions of the invention may comprise one or more further therapeutic agents. Examples of further therapeutic agents that may be present in a composition of the present invention include, but not limited to, aminosalicylates (for example, mesalazine, olsalazine, sulphasalazine, balsalazide), corticosteroids (for example, prednisolone, prednisone, methylprednisolone, budesonide, hydrocortisone and beclometasone dipropionate), immunosuppressants (for example, azathioprine, mercaptopurine, methotrexate, ciclosporin and tacrolimus), anti-TNF drugs (for example, infliximab, adalimumab and golimumab), antibiotics (for example, ciprofloxacin and metronidazole), anti-integrin drugs (for example, vedolizumab and natalizumab), interleukin inhibitors (for example, ustekinumab) and Janus Kinase inhibitors (for example, tofacitinib, filgotinib, upadacitinib, and TYK2 inhibitors such as BMS-986165).

Inflammatory Bowel Diseases

The compounds of formula (I), and pharmaceutical compositions of the invention, find use in treating IBDs, for example CD and UC.

As such, a compound of formula (I) for use according to the invention, or composition of the invention, may be administered to a subject having an IBD, such as CD or UC. The subject may be a human subject, for example a human patient.

The subject may have an IBD that may be classed as refractory, relapsed or refractory-relapsed. For example, the subject may have refractory, relapsed or refractory-relapsed CD or UC. Additionally, or alternatively, the subject may have an IBD that is partially or completely resistant to established IBD treatments, such as aminosalicylates and corticosteroids. For example, the IBD may be CD or UC that is partially or completely resistant to aminosalicylate and/or corticosteroid treatment or prophylaxis. Additionally, or alternatively, the subject may be one who has experienced, or at risk of experiencing, an adverse reaction to an established IBD treatment, such as aminosalicylates and corticosteroids.

The compounds of formula (I) for use according to the invention, and compositions of the invention, may be administered to a subject known or suspected of being at risk of developing an IBD. For example, subjects with a known or suspected genetic predisposition for developing an IBD, such as CD or UC. For example, the compounds of formula (I), or composition of the invention, may be administered to a subject in need of extended remission of an IBD and/or slower progression of an IBD.

The compounds of formula (I) and compositions of the invention find utility in a method of treating or preventing an IBD, said method comprising a step of administering a compound of formula (I), or a composition of the invention, to a subject having an IBD, such as CD or UC. In certain embodiments, the method of treating or preventing an IBD comprises a step of administering a compound of formula (I), or a composition of the invention, to a subject known or suspected of being at risk of developing an IBD.

In certain embodiments, the method of treatment or prophylaxis comprises a step of delivering a compound of formula (I), or a pharmaceutical composition of the invention, to the small and/or large intestine of a subject. For example, a step of delivering a compound of formula (I), or a pharmaceutical composition of the invention, to one or more of the duodenum, jejunum and ileum; and/or one or more of the caecum, ascending colon, traverse colon, descending colon and/or sigmoid colon. The method of treatment or prophylaxis may also comprise a step of orally or rectally administering a compound of formula (I), or a composition of the invention, to a subject.

The compounds of formula (I) also find use in the manufacture of a medicament for the treatment or prophylaxis of an IBD. For example, the compounds of formula (I) may be used in the manufacture of a medicament for the treatment or prophylaxis of CD or UC.

Delivery to the Small and/or Large Intestine

The composition according to the invention may be adapted for selective release of the compound of formula (I) in the small intestine or the large intestine following rectal or oral administration. For example, in certain embodiments, the compound of formula (I), or pharmaceutical composition of the invention, is administered locally to the small and/or large intestine. This may be accomplished by the use of particular coatings and/or formulations.

The compositions of the invention may have an enteric coating. Enteric coatings which protect the active ingredients in a composition from attack and degradation in the stomach, and permit release within the intestines, are known. The optimal coating for any particular formulation depends on the exact intended use, and coatings may be tailored to release the active ingredient in a particular region of the intestines, or at a particular time following ingestion.

The composition of the invention may be adapted to release the compound of formula (I) in the small intestine, for example, in one or more of the duodenum, jejunum and ileum. Additionally, or alternatively, the composition of the invention may be adapted to release the compound of formula (I) in the large intestine, for example, in one or more of the caecum, ascending colon, traverse colon, descending colon and/or sigmoid colon.

The composition of the invention may be in a solid or semi-solid form, preferably comprising an enteric coating, adapted to release the compound of formula (I) in the small intestine and/or large intestine. Such a formulation may contain one or more intermediate layers between the active ingredient and the outer enteric coating. In certain embodiments, it is possible for a composition of the invention to release a portion of its contents at one or more particular regions of the small intestine, and a further portion of its contents in one or more particular regions of the large intestine.

Dosage Regimens

The amount of a compound of formula (I) which is required to achieve a therapeutic effect will vary with particular route of administration and the characteristics of the subject under treatment, for example the species, age, weight, sex, medical conditions, the particular IBD and its severity, and other relevant medical and physical factors. An ordinarily skilled physician can readily determine and administer an effective amount of the compound of formula (I) required for treatment or prophylaxis of the IBD.

The compound of formula (I) may be administered daily (including several times daily), every second or third day, weekly, every second, third or fourth week or even as a high single dose depending on the subject and IBD to be treated.

Preferably, the compound of formula (I) (excluding the mass of any counterion or solvent), may be administered in an amount of about 1 to 1000 mg per administration. For example, 1, 5, 10, 15, 20, 25, 40, 50, 60, 70, 80, 90, 100, 110, 120, 130, 140, 150, 200, 300, 400, 500, 600, 700, 800, 900 and 1000 mg.

In certain embodiments, the compound of formula (I) is administered as a composition. Preferably, the composition is a pharmaceutical composition of the present invention.

Whilst a compound of formula (I) may be used as the sole active ingredient in the present invention, it is also possible for it to be used in combination with one or more further therapeutic agent(s), and the use of such combinations provides one embodiment of the invention. Such further therapeutic agents may be agents useful in the treatment or prophylaxis of an IBD, or other pharmaceutically active materials. Such agents are known in the art. Examples of further therapeutic agents for use in the present invention include those described herein.

The one or more further therapeutic agent(s) may be used simultaneously, sequentially or separately with/from the administration of the dosage a compound of formula (I). The individual components of such combinations can be administered separately at different times during the course of therapy or concurrently in divided or single combination forms. An ordinarily skilled physician can readily determine and administer the effective amount of one or more therapeutic agent required to have the desired therapeutic effect.

The compound of formula (I), or salt or solvate thereof, may be administered as an oral or rectal dosage, and thus the dosage of the compound of formula (I) must be in the form suitable for delivery of the compound of formula (I) to the small and/or large intestine.

Preferred unit dosage compositions for use according to the invention are those containing an effective dose, or an appropriate fraction thereof, of the compound of formula (I). The release of the compound of formula (I) from certain composition may also be sustained, for example, if the composition contains suitable controlled-release excipients.

Kits

The present invention provides a kit comprising a compound of formula (I), one or more pharmaceutically acceptable excipients, and optionally one or more further therapeutic agents that are useful in the treatment or prophylaxis of an IBD. Examples of such further therapeutic agents include those described herein as being suitable for use in the present invention, and being optionally present in a pharmaceutical composition of the invention as a further therapeutic agent.

Kits of the present invention find use in the treatment and prophylaxis of an IBD, especially CD and UC.

For the avoidance of doubt, the compound of formula (I) present in a kit according to the present invention is in a form and quantity suitable for use according to the present invention. Suitable pharmaceutical compositions and formulations are described herein. The skilled person can readily determine a quantity of the compound of formula (I) suitable for including in a kit of the invention, and for use according the invention.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

### Example 1: Synthesis of 5-chloro-4-hydroxy-1-methyl-2-oxo-N-phenyl-quinoline-3-carboxamide Free Acid (Example Compound 1)

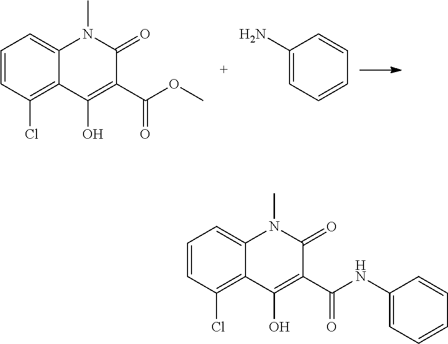 [SMILES: COC(=O)c1c(O)c2c(Cl)cccc2n(C)c1=O ; Nc1ccccc1 ; Cn1c(=O)c(C(=O)Nc2ccccc2)c(O)c2c(Cl)cccc21]

A mixture of methyl 5-chloro-4-hydroxy-1-methyl-2-oxo-quinoline-3-carboxylate (25 g, 0.0934 mol) and aniline (17.4 g, 0.0333 mol, 2 eq) in Toluene (600 mL) were stirred at 100° C. for 17 h. HPLC revealed total conversion to product. The reaction was removed from heating and the product precipitated. The reaction was left to stand at room temperature for two days. The soft solid cake was suspended in n-heptane (500 mL) and after 5 minutes of stirring the solid was filtered off. The solid was washed with a 1:1 mixture of toluene and n-heptane (1000 mL) to give crude 5-chloro-4-hydroxy-1-methyl-2-oxo-N-phenyl-quinoline-3-carboxamide. The product was recrystallized from toluene and washed with heptane and then further purified by column chromatography (petroleum ether 100%->DCM 100%). Final recrystallization from AcCN gave the desired product (21.5 g, 70% yield).

LC/MS: M+H=329.10. ¹H NMR (400 MHz, DMSO-d₆) δ: 12.75 (1H, s), 7.73 (1H, m), 7.65 (3H, m), 7.44 (3H, m), 7.22 (1H, m), 3.70 (s, 3H).

### Example 2: Synthesis of 5-chloro-4-hydroxy-1-methyl-2-oxo-N-phenyl-quinoline-3-carboxamide Potassium Salt (Example Compound 2)

Example compound 1 (300 mg) was suspended in ethanol (6.0 mL) and 5 M aqueous potassium hydroxide (0.198 mL, ˜ 1.1 eq) was added. The resulting suspension was shaken well by hand and then agitated and temperature-cycled between 40° C. and ambient for 48 hours.

The product was isolated by filtration, washed with ethanol (2×1 mL) and dried at 45° C. under vacuum to constant weight. White crystals were obtained. The yield was 251 mg.

### Example 3: Biological activity of Example compound 1 in an Ulcerative Colitis Mouse Model

Ulcerative Colitis was modelled in C57Bl/6 mice by administering Dextran Sulphate Sodium (DSS) in drinking water (1.5% w/v) for 5 days. Mice were monitored daily for weight loss and clinical symptoms of disease for 10 days starting at the beginning of DSS administration.

Animals in different treatment groups were administered either vehicle, example compound 1 (1 mg/kg as a 0.1 mg/mL aqueous suspension with CMC-Na (sodium carboxymethyl cellulose, 2%, w/v), or anti-TNFα antibody (anti-mouse TNFα antibody clone XT3.11). One control group of mice did not receive DSS, example compound 1 or vehicle (herein referred to as “naïve animals”). Vehicle and example compound 1 were administered as daily administrations starting on either Day −7, though day 9. Anti-TNFα antibody was administered at 500 μg/treatment on Days 0, 2, 4, and 6. Following termination on Day 10, colons were removed and measured. The length and weight of the colon were also assessed.

Compared to naïve animals, animals given DSS water developed clinical signs of disease including weight loss and diarrhoea as well as gross pathological signs of disease on experiment termination. Additionally, colon length was significantly reduced in diseased animals compared to naïve animals, while weight was increased. Shortened length and increased weight:length ratio indicates edema associated with ulcerative colitis.

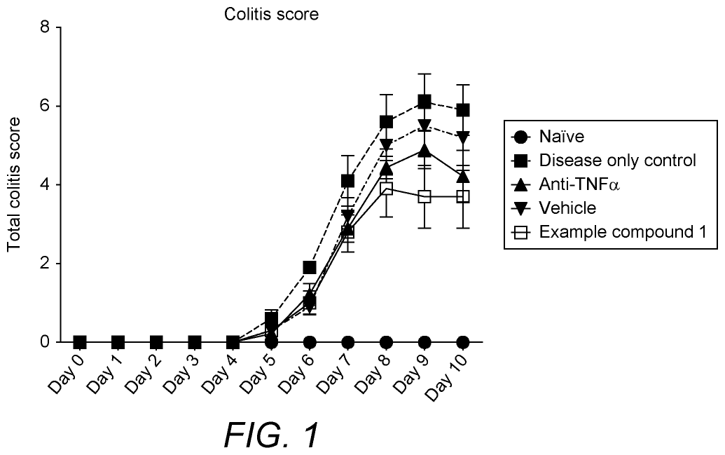

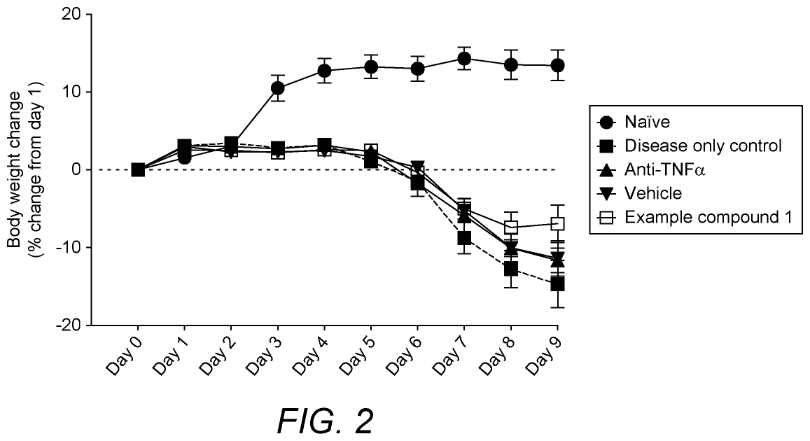

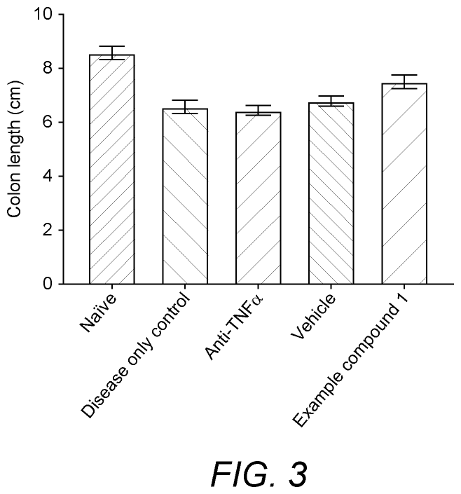

As shown in FIGS. 1 to 3, daily oral treatment with example compound 1 resulted in a significant reduction in colitis clinical score on Day 9 of the study, reduced weight loss, and a significant increase in colon length on experiment termination compared to vehicle-treated controls, indicating an efficacious effect of the treatment.

### Example 4: Biological Activity of Example Compound 2

### Example 4 a): Activity of Example Compound 2 in Inhibiting Inflammation/Edema Associated with Inflammatory Bowel Disease

The efficacy of Example compound 2 in inhibiting inflammation/edema associated with inflammatory bowel disease was evaluated in a CD4+ adoptive transfer induced inflammatory bowel disease in mice.

The details of the mice were as follows:

Species/Strain or Breed: Fox Chase C.B-17 SCID & Balb/C

Vendor: Charles River

Age/Wt at Arrival: CB-17 SCID—6-7 weeks old
Balb/C—11-12 weeks old

Gender: Female

Acclimation: Acclimatization for at least 7 days after arrival

Housing: 5 animals/cage

On study day −1, SCID mice were weighed and evenly distributed into treatment groups based on body weight.

On study day 0, Balb/C mice were terminated, and spleens obtained for CD4⁺CD45RB^{high} cell isolation (Using SCID IBD Cell Separation Protocol). After cells were sorted and obtained, each animal in the treatment groups received an IP injection of CD4⁺CD45RB^{high} cells at a minimum 4×10⁵ cells (200 μl/mouse injections). A naïve group of mice was followed through the experiment without receiving the injection of cells. The naïve group comprised 5 animals.

On study day 21, treatment with Example compound 2 (1 mg/kg, daily day 21 to 49) was initiated. The compound was formulated as a 0.1 mg/mL suspension with sodium carboxymethyl cellulose (1%, w/v). Mice received either Example compound 2 or vehicle. Each of those groups comprised 10 animals.

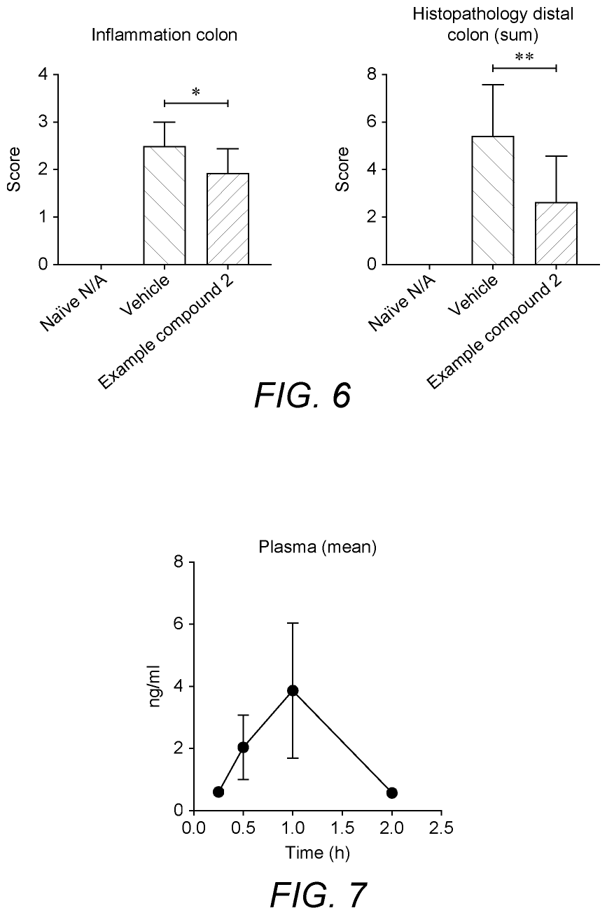

On study day 49, animals were anesthetized with Isoflurane and bled to exsanguination followed by cervical dislocation. The entire colon was removed, measured, and weighed. The colon was analysed for its level of interferon-γ and IL-22. The results are shown in FIG. 5. The inflammation in the colons was also scored by visual and histopathological assessments. The scores are shown in FIG. 6.

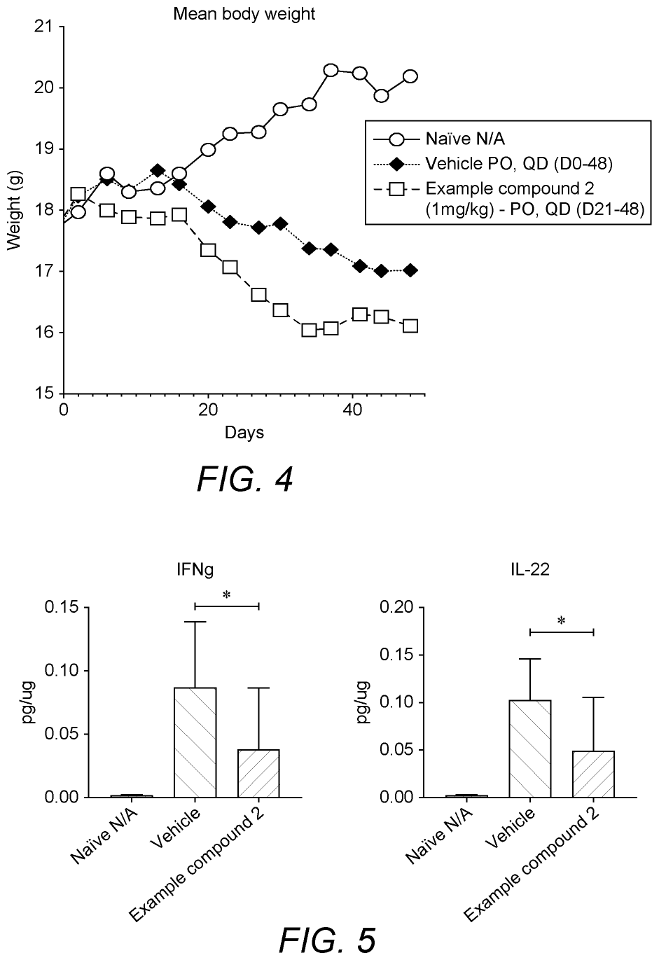

Each animal was weighed at 3- or 4 day intervals and the mean body weight of the mice in the three groups is shown in FIG. 4. As can be seen in the figure, the mice treated with Example compound 2 have less weight loss than mice treated with vehicle only. In FIG. 5, it is seen that the mice treated with Example compound 2 had lower levels of inflammatory markers than the mice treated with vehicle only. Similarly, it is seen in FIG. 6 that the mice treated with Example compound 2 had fewer signs of inflammation than the mice treated with vehicle only (* indicates a statistical significance of p<0.05; ** indicates a statistical significance of p<0.01). The results in FIGS. 5 and 6 demonstrate that Example compound 2 has a local anti-inflammatory effect in the colon of the animals.

### Example 4 b): Activation of the Aryl Hydrocarbon Receptor (AhR) in the Colon of Mice Following Administration of Example Compound 2

The potential of Example compound 2 to activate the aryl hydrocarbon receptor (AhR) in the colon following oral administration was evaluated in a wild-type (WT) mice.

The details of the mice were as follows:

Number of animals: Order 51 (50 Study+1 Extra)

Species/Strain or Breed: C57Bl/6

Vendor: Taconic

Age/Wt at Arrival: 6-7 weeks

Gender: Female

Housing: 5 animals/cage

On study day 0, animals were weighed and evenly distributed into treatment groups based on body weight. Also, on study day 0, treatments were initiated (a summary of the treatment schedule is shown in the table below). On study day 14, animals were asphyxiated via CO₂ inhalation followed by cervical dislocation. The entire colon was removed, collected and prepared for qPCR analysis of CYP1A1 (Normalized to GAPDH & ACTB).

| | | | Dose | | | | | Dose |
|---|---|---|---|---|---|---|---|---|
| | | | Level | Dose | | Dosing | Dose Vol | Conc |
| Group | N¹ | Treatment² | (mg/kg) | Route³ | Regimen⁴ | Days | (mL/kg) | (mg/mL) |
| 1 | 4 | Naive | N/A | N/A | N/A | N/A | N/A | N/A |
| 2 | 4 | Ex 2 | 1.0 | PO | QD | DO-13 | 10 | 0.1 |
| | | | mg/kg | | | | | |
| 3 | 4 | Ex 2 | 0.1 | PO | QD | DO-13 | 10 | 0.01 |
| | | | mg/kg | | | | | |
| 4 | 4 | Ex 2 | 0.01 | PO | QD | DO-13 | 10 | 0.001 |
| | | | mg/kg | | | | | |

¹N, number of mice in the group.
²Naïve mice received no test compound or vehicle. Ex 2 = Example compound 2.
³PO, per os (oral administration).
⁴QD, quaque die (once a day).

The Example compound 2 was formulated as a 0.1 mg/ml, 0.01 mg/ml or 0.001 mg/ml suspension with sodium carboxymethyl cellulose (1%, w/v).

CYP1A1 qPCR Results

| | | Colon CYP1A1 | | Liver CYP1A1 | |
|---|---|---|---|---|---|
| | | (fold change | | (fold change | |
| | Dose | over untreated) | | over untreated) | |
| Group | (mg/kg) | Mean | SE | Mean | SE |
| 2 | 1.0 | 8.34 | 2.75 | 3.44 | 0.30 |
| 3 | 0.1 | 7.42 | 2.62 | 2.81 | 0.65 |
| 4 | 0.01 | 1.03 | 0.34 | 1.08 | 0.34 |

As can be seen in the table, WT mice treated with the Example compound 2 salt at 1.0 and 0.1 mg/kg had a significant increase in CYP1A1 mRNA expression compared to untreated animals indicating AhR activation. Furthermore, the results demonstrate that the Example compound 2 has a local AhR activating effect as the increase in liver CYP1A1 expression was lower than the increase in CYP1A1 in the colon.

### Example 4 c): In Vivo Pharmacokinetics of Example Compound 2

An in vivo pharmacokinetic study in rats was performed to determine if Example compound 2 can be absorbed and detected systemically after oral dosing. 4 male Sprague Dawley rats, approximately 225 g-250 g at dosing were dosed with 1 mg/kg of Example compound 2 formulated as a 0.1 mg/mL suspension with sodium carboxymethyl cellulose (1%, w/v). Blood samples were taken at 15 min, 30 min, 1 h, 2 h, 4 h, 6 h, 8 h and 24 h post dose

At each blood sampling approximately 250 μL blood was sampled in K3EDTA vials and approximately 100 μL plasma was also prepared.

The plasma samples were prepared by mixing 50 μL plasma with 250 μL of internal standard solution (20 ng/mL of phenacetin in ACN with 1% formic acid), mixed and centrifuged (20 min, 4000 rpm).

Plasma samples were transferred to Waters Ostro 96-well plate and drawn through the plate by applying 6-8 psi positive pressure for 10 min. 100 μL of supernatant was further diluted with 50 μL UP water and sample was submitted to analysis.

The standard and QC samples were prepared into blank rat colon homogenate and blank rat plasma. Standards were spiked into concentrations 0.1-10000 ng/mL of the analytes, QC samples into 3, 30, 300 and 3000 ng/mL, and were otherwise treated as samples.

The level of Example compound 2 in the plasma at the various time points is shown in FIG. 7. It is seen that the compound is detected in the plasma and that it is rapidly cleared.

1. A compound according to formula (I): 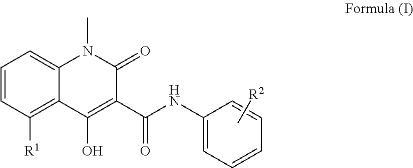
or a tautomer thereof,
wherein,
R¹ is hydrogen, hydroxyl, C₁₋₃alkyl, OC₁₋₃alkyl or halogen; and
R² is hydrogen, hydroxyl, OC₁₋₃alkyl, halogen or C₁₋₃alkyl optionally substituted with at least one halogen;
or a pharmaceutically acceptable salt or solvate thereof;
for use in the treatment or prophylaxis of an inflammatory bowel disease.

2. The compound for use as claimed in claim 1, wherein the compound for use is the compound of formula (Ia): 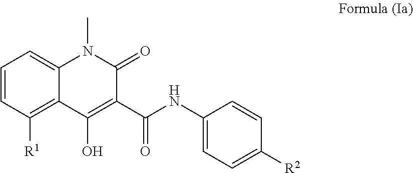
or a tautomer thereof.

3. The compound for use as claimed in claim 1 or 2, wherein R² is hydrogen.

4. The compound for use as claimed in claim 3, wherein R¹ is hydrogen or halogen.

5. The compound for use as claimed in claim 1 or 2, wherein the compound is selected from:

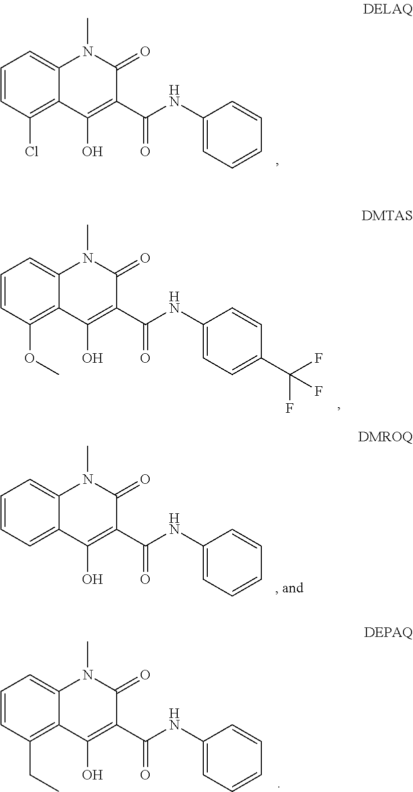 [SMILES: Cn1c(=O)c(C(=O)Nc2ccccc2)c(O)c2c(Cl)cccc21 ; COc1cccc2c1c(O)c(C(=O)Nc1ccc(C(F)(F)F)cc1)c(=O)n2C ; Cn1c(=O)c(C(=O)Nc2ccccc2)c(O)c2ccccc21 ; CCc1cccc2c1c(O)c(C(=O)Nc1ccccc1)c(=O)n2C]

6. The compound for use as claimed in claim 5, wherein the compound is selected from:

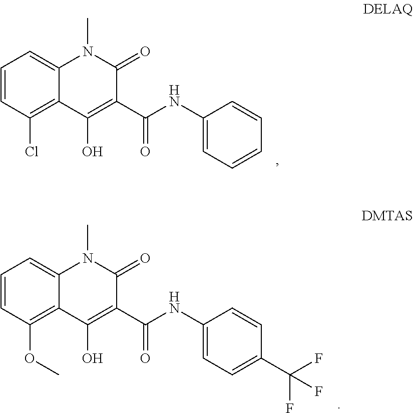 [SMILES: Cn1c(=O)c(C(=O)Nc2ccccc2)c(O)c2c(Cl)cccc21 ; COc1cccc2c1c(O)c(C(=O)Nc1ccc(C(F)(F)F)cc1)c(=O)n2C]

7. The compound for use as claimed in any one of claims 1 to 6, wherein the inflammatory bowel disease is Ulcerative Colitis or Crohn's disease.

8. The compound for use as claimed in any one of claims 1 to 7, wherein the compound is administered locally to the small and/or large intestine.

9. A pharmaceutical composition comprising a compound of formula (I) as defined in any one of claims 1 to 6 and at least one pharmaceutically acceptable excipient, wherein said pharmaceutical composition has a solid or semi-solid form adapted for release of the compound in the small and/or large intestine.

10. The pharmaceutical composition as claimed in claim 9, wherein said pharmaceutical composition comprises an enteric coating.

11. The pharmaceutical composition as claimed in claim 9 or 10, wherein the pharmaceutical composition contains less than 10 mole percent (mol %) of a precursor of the compound of formula (I), wherein the mol % is the proportion of compound present in the composition relative to the total combined number of moles of compound of formula (I) and the precursor present in the composition.

12. The pharmaceutical composition as claimed in claim 11, wherein said pharmaceutical composition is substantially free from the precursor of the compound of formula (I).

13. The pharmaceutical composition as claimed in claim 11 or 12, wherein said precursor of the compound of formula (I) is a compound of formula (II): 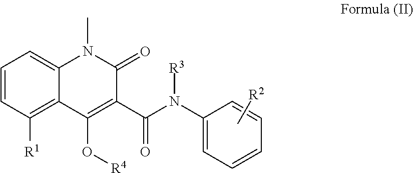
or a tautomer thereof,
wherein,
R¹ is hydrogen, hydroxyl, C₁₋₃alkyl, OC₁₋₃alkyl or halogen;
R² is hydrogen, hydroxyl, OC₁₋₃alkyl, halogen or C₁₋₃alkyl optionally substituted with at least one halogen;
R³ and R⁴ are independently selected from hydrogen, C(O)H, C(O)methyl, C(O)ethyl, C(O)propyl, C(O)CH(CH₃)₂, C(O)C(CH₃)₃, C(O)phenyl, C(O)CH₂phenyl, CO₂H, CO₂CH₃, CO₂CH₂CH₃, CO₂CH₂phenyl, C(O)NHCH₃, C(O)N(CH₃)₂, C(O)NHCH₂CH₃, C(O)N(CH₂CH₃)₂, C(O)NHphenyl, C(O)NHCH₂phenyl, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising methyl, ethyl, OCH₃, OCH₂CH₃, SCH₃, S(O)CH₃, S(O)₂CH₃, S(O)₂N(CH₃)₂, CF₃, OCF₃, F, Cl, OH, CO₂H, CO₂CH₃, CO₂CH₂CH₃, C(O)NH₂, C(O)N(CH₃)₂, NH₂, NH³⁺, N(CH₃)₂, NCH₃³⁺, NHC(O)CH₃, NC(═NH)NH₂, OS(O)₂OH, S(O)₂OH, OP(O)(OH)₂, and P(O)(OH)₂, provided that R³ and R⁴ are not both hydrogen; or wherein R³ is hydrogen, R⁴ is P(O)(OH)₂, P(O)(OCH₃)₂, P(O)(OCH₂CH₃)₂, P(O)(OPhenyl)₂, P(O)(OCH₂Phenyl)₂, S(O)₂OH, S(O)₂NH₂ or S(O)₂N(CH₃)₂;
or a pharmaceutically acceptable salt or solvate thereof.

14. The pharmaceutical composition as claimed in claim 13, wherein the compound of formula (II) is the compound of formula (IIa):

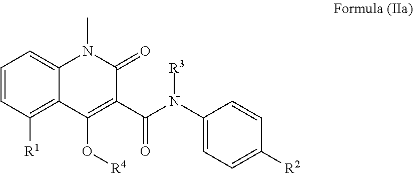

15. The pharmaceutical composition as claimed in claim 13 or 14, wherein R¹ and R² of the compound of formula (II) present in the pharmaceutical composition are the same as R¹ and R² of the compound of formula (I) present in the pharmaceutical composition.

16. The pharmaceutical composition as claimed in any one of claims 9 to 15, further comprising one or more further therapeutic agent.

17. The pharmaceutical composition as claimed in any one of claims 9 to 16, for use in the treatment or prophylaxis of an inflammatory bowel disease, for example Ulcerative Colitis and Crohn's disease.

18. Use of a compound of formula (I) as defined in any one of claims 1 to 6 in the manufacture of a medicament for the treatment or prophylaxis of an inflammatory bowel disease.

19. A method of treating or preventing an inflammatory bowel disease comprising a step of administering to a subject in need thereof a compound of formula (I) as defined in any one of claims 1 to 6, or the pharmaceutical composition as claimed in any one of claims 9 to 16.

20. The method as claimed in claim 19, or the use as claimed in claim 18, wherein the inflammatory bowel disease is Crohn's disease or Ulcerative Colitis.

21. The method as claimed in claim 19 or 20, comprising a step of delivering the compound of formula (I), or the pharmaceutical composition, to the small and/or large intestine of the subject.

22. The method as claimed in any one of claims 19 to 21, comprising a step of orally or rectally administering the compound of formula (I), or the pharmaceutical composition, to the subject.
